(19) United States
(12) Patent Application Publication
(10) Pub. No.: US 2011/0257562 A1
(43) Pub. Date: Oct. 20, 2011

(54) METHOD AND APPARATUS EMPLOYING ULTRASOUND ENERGY TO REMODULATE VASCULAR NERVES

(76) Inventor: Alan Schaer, San Jose, CA (US)

(21) Appl. No.: 13/076,234

(22) Filed: Mar. 30, 2011

Related U.S. Application Data
(60) Provisional application No. 61/320,219, filed on Apr. 1, 2010.

(51) Int. Cl. A61N 7/00

(52) U.S. Cl. 601/2

(57) ABSTRACT

Methods and apparatus for treating hypertension and other vessel dilation conditions provide for delivering acoustic energy to a vascular nerve to remodel the tissue and nerves surrounding the vessel. In the case of treating hypertension, a catheter carrying an ultrasonic or other transducer is introduced to the renal vessel, and acoustic energy is delivered to the tissue containing nerves to remodel the tissue and remodulate the nerves.

### CROSS-REFERENCES TO RELATED APPLICATIONS

The present application claims the benefit of U.S. Provisional Patent Application No. 61/320,219 (Attorney Docket No. 021574-000400US), filed Apr. 1, 2010, the entire contents of which are incorporated herein by reference.

### BACKGROUND OF THE INVENTION

1. Field of the Invention

In a general sense, the invention is directed to systems and methods for remodulating vascular nerves. More specifically, the invention is directed to systems and methods for treating hypertension mediated by conduction within the vascular nerves, particularly those surrounding the renal arteries.

2. Description of the Background Art

Congestive Heart Failure (“CHF”) is a condition that occurs when the heart becomes damaged and reduces blood flow to the organs of the body. If blood flow decreases sufficiently, kidney function becomes altered, which results in fluid retention, abnormal hormone secretions and increased constriction of blood vessels. These results increase the workload of the heart and further decrease the capacity of the heart to pump blood through the kidneys and circulatory system.

It is believed that progressively decreasing perfusion of the kidneys is a principal non-cardiac cause perpetuating the downward spiral of CHF. Moreover, the fluid overload and associated clinical symptoms resulting from these physiologic changes result in additional hospital admissions, poor quality of life and additional costs to the health care system.

In addition to their role in the progression of CHF, the kidneys play a significant role in the progression of Chronic Renal Failure (“CRF”), End-Stage Renal Disease (“ESRD”), hypertension (pathologically high blood pressure) and other cardio-renal diseases. The functions of the kidneys can be summarized under three broad categories: filtering blood and excreting waste products generated by the body's metabolism; regulating salt, water, electrolyte and acid-base balance; and secreting hormones to maintain vital organ blood flow. Without properly functioning kidneys, a patient will suffer water retention, reduced urine flow and an accumulation of waste toxins in the blood and body. These conditions result from reduced renal function or renal failure (kidney failure) and are believed to increase the workload of the heart. In a CHF patient, renal failure will cause the heart to further deteriorate as fluids are retained and blood toxins accumulate due to the poorly functioning kidneys.

It has been established in animal models that the heart failure condition results in abnormally high sympathetic activation of the kidneys. An increase in renal sympathetic nerve activity leads to decreased removal of water and sodium from the body, as well as increased renin secretion. Increased renin secretion leads to vasoconstriction of blood vessels supplying the kidneys, which causes decreased renal blood flow. Reduction of sympathetic renal nerve activity, e.g., via denervation, may reverse these processes.

Prior art therapies for vessel ablation require direct electrode contact with the vessel wall. This can lead to excessive heating at the electrode-tissue interface. Even when cooling of an electrode (e.g., RF electrode) is attempted, it is difficult to ensure sufficient uniform cooling over the entire surface of the electrode, leaving risk of damage to the inner tissue layer(s) (e.g., in arteries, the intima and/or media layers). If aggressive RF cooling is achieved at the tissue surface, too much energy density may be required at the greater depths, leading to uncontrolled superheating, or “pops” in tissue that can lead to vessel rupture. As the nerves and tissues of interest are beyond the inner layers, the cooling must be strong enough at the surface and energy absorption slow enough deeper in the tissue to allow protection of the inner layer(s) while achieving reliable and safe remote heating. Ultrasound can provide such a benefit. However, ultrasound transducers can be inefficient at converting electrical energy to acoustic energy, with the byproduct being heat. Thus for an ultrasound transducer to produce sufficient acoustic energy for heating at the desired tissue depth, it must be designed and mounted in such a way as to prevent excessive heat buildup. It must also have a means for adequately removing any heat generated by the transducer that could be conducted to the tissue, as well as removing heat from acoustic absorption by the tissue at the luminal surface. Of particular concern is heating the arterial intima and/or media to the point at which surface disruption and/or necrosis occurs, leading to acute or chronic vessel stenosis. High Intensity Focused Ultrasound (HIFU) has the benefit of sparing regions of tissue from heating that do not require therapy (e.g., the artery intima and more remote tissue structures). However, the focal region location and/or energy density may be difficult to control and monitor, increasing the risk of tissue overheating. Renal arteries average about 5 mm in diameter, which is smaller than many luminal applications of ultrasound in the prior art. The present invention addresses these challenges.

In view of the foregoing, and notwithstanding the various efforts exemplified in the prior art, there remains a need for a more simple, rapid, minimally invasive, and more effective approach to treating vascular nerves from an intra-vascular approach that minimizes risk to the patient.

### BRIEF SUMMARY OF THE INVENTION

The present invention seeks to heat nerves surrounding a blood vessel using ultrasound energy. The preferred method is to use ultrasound energy to heat the outer vascular tissue layers and extra-vascular tissue containing nerve pathways, and thus create necrotic and/or ischemic regions in this tissue. The lesions interrupt or remodulate nerve pathways responsible for vasoconstriction. In general, during the heating process, the invention employs means to minimize heat damage to the intima and/or media layer of the vessel that could lead to vessel stenosis and/or thrombosis. Ultrasound may also be used (continuously or in pulsed mode) to create shock waves that cause mechanical disruption through cavitation that create the desired tissue effects. While this invention relates broadly to many vascular regions in the body, the focus of the disclosure will be on the treatment of renal vessels.

The key advantage of an ultrasound ablation system over others is that a uniform annulus of tissue can be heated simultaneously. Alternatively, the transducers can be designed so that only precise regions of the circumference are heated. Ultrasound also penetrates tissue deeper than radiofrequency (RF) or simple thermal conduction, and therefore can be delivered with a more uniform temperature profile. Thus lesions can be created at deeper locations than could be safely achieved with RF electrodes inside the vessel, or RF needles puncturing the tissue. Similarly, the deeper heating and uniform temperature profile also allow for an improved ability to create a cooling gradient at the surface. Relatively low power can be delivered over relatively long durations to maximize tissue penetration but minimize surface heating. A device using ultrasound for ablation may also be configured to allow diagnostic imaging of the tissue to determine the proper location for therapy and to monitor the lesion formation process.

In a first specific aspect of the present invention, methods for remodeling vascular tissue comprise positioning a transducer at a target site in a vessel of a patient. The transducer is energized to produce acoustic energy under conditions selected to induce tissue remodeling in at least a portion of the tissue circumferentially surrounding the vessel. In particular, the tissue remodeling may be directed at or near the luminal surface, but will more usually be directed at a location at a depth beneath the luminal surface, typically from 1 mm to 10 mm, more usually from 2 mm to 6 mm. In the most preferred cases, the tissue remodeling will be performed in a generally uniform matter on a ring or region of tissue circumferentially surrounding the vessel, as described in more detail below.

The acoustic energy will typically be ultrasonic energy produced by electrically exciting an ultrasonic transducer which may optionally be coupled to an ultrasonic horn, resonant structure, or other additional mechanical structure which can focus or enhance the acoustic energy. In an exemplary case, the transducer is a phased array transducer capable of selectively focusing and/or scanning energy circumferentially around the vessel.

The acoustic energy is produced under conditions which may have one or more of a variety of biological effects. In many instances, the acoustic energy will be produced under conditions which interrupt, remodulate, or remodel nerve pathways within the tissue, such as the sympathetic renal nerves as described in more detail hereinafter. The acoustic energy may also remodel biochemical processes within the tissue that contribute to vessel constriction signaling. The initial dessication and shrinkage of the tissue, followed by the healing response may serve to stretch and/or compress the incident and surrounding nerve fibers, which contributes to nerve remodulation.

Preferred ultrasonic transducers may be energized to produce unfocused acoustic energy in the range from 10 W/cm² to 100 W/cm², usually from 30 W/cm² to 70 W/cm². The transducer will usually be energized at a duty cycle in the range from 10% to 100%, more usually from 70% to 100%. Focused ultrasound may have much higher energy densities, but will typically use shorter exposure times and/or duty cycles. For tissue heating, the transducer will usually be energized under conditions which cause a temperature rise in the tissue to a tissue temperature in the range from 55° C. to 95° C., usually from 60° C. to 80° C. In such instances, particularly when ultrasound is not focused, it will usually be desirable to cool the luminal surface, (e.g., intima layer within an artery).

Usually, the transducer will be introduced to the vessel using a catheter which carries the transducer. In certain specific embodiments, the transducer will be carried within an inflatable balloon on the catheter, and the balloon when inflated will at least partly engage the luminal wall in order to locate the transducer at a pre-determined position relative to the luminal target site. In a particular instance, the transducer is disposed within the inflatable balloon, and the balloon is inflated with an acoustically transmissive material so that the balloon will both center the transducer and enhance transmission of acoustic energy to the tissue. In an alternative embodiment, the transducer may be located between a pair of axially spaced-apart balloons. In such instances, when the balloons are inflated, the transducer is centered within the lumen. Usually, an acoustically transmissive medium is then introduced between the inflated balloons to enhance transmission of the acoustic energy to the tissue. In any of these instances, the methods of the present invention optionally comprise moving the transducer relative to the balloons, typically in an axially direction, in order to focus or scan the acoustic energy at different locations on the luminal tissue surface.

In specific embodiments, the acoustically transmissive medium may be cooled in order to enhance cooling of the luminal tissue surface. Additionally, the methods may further comprise monitoring temperature of the luminal tissue surface and/or at a point beneath the luminal tissue surface.

In other specific examples, methods of the present invention further comprise focusing acoustic energy beneath the luminal tissue surface. In such instances, focusing may be achieved using a phased array (by selectively energizing particular elements of the array) and the tissue may be treated at various locations and various depths.

The methods as described above are particularly preferred for treating patients suffering from hypertension where the acoustic energy remodels the outer vessel and extra-vascular tissue.

The present invention still further comprises an apparatus for remodeling the outer vessel and extra-vascular tissue. Such an apparatus comprises a catheter adapted to be intravascularly introduced to a renal vessel and a transducer on the catheter. The transducer is adapted to deliver acoustic energy to the vessel tissue in order to reduce hypertension.

Specific apparatus constructions include providing an inflatable balloon on the catheter, where the balloon is adapted when inflated to position the catheter within the vessel so that the transducer can deliver energy to the vessel tissues. The transducer is usually positioned co-axially within the balloon, and means may be provided for inflating the balloon with an acoustically transmissive medium.

Alternatively, the transducer may be positioned between a pair of axially-spaced-apart balloons, where the apparatus will typically further comprise means for delivering an acoustically transmissive medium between the balloons. In all instances, the apparatus may further comprise means for cooling the acoustically transmissive medium, and means for axially translating the transducer relative to the catheter. In certain specific examples, the transducer comprises a phased array transducer.

### BRIEF DESCRIPTION OF THE DRAWINGS

FIG. 1 is an illustration of the tissue structures comprising the renal vessels.

FIG. 2 is an Ultrasound Ablation System for Hypertension Treatment.

FIG. 3 is an Ultrasound Ablation Catheter.

FIGS. 4a-c is a renal vessel with different lesion patterns

FIG. 5 is a cylindrical PZT material.

FIG. 6 is an annular array of flat panel transducers and the acoustic output from the array.

FIGS. 7a-7d is isolated active sectors of a transducer formed by isolating the plated regions.

FIG. 8 is a selective plating linked with continuous plating ring.

FIG. 9 is a cylindrical transducer with non-resonant channels.

FIG. 10 is a cylindrical transducer with an eccentric core.

FIG. 11 is a cylindrical transducer with curved cross-section and resulting focal region of acoustic energy.

FIG. 12 is an illustration of acoustic output from conical transducers.

FIGS. 13a and 13b is a longitudinal array of cylindrical transducers.

FIG. 14 is a transducer mounting configuration using metal mounts.

FIG. 15 shows transducer geometry variations used to enhance mounting integrity.

FIG. 16 is transducer plating variations used to enhance mounting integrity.

FIG. 17 shows cooling flow through the catheter center lumen, exiting the tip.

FIG. 18 shows cooling flow recirculating within the catheter central lumen.

FIG. 19 shows cooling flow circulating within the balloon.

FIG. 20 shows cooling flow circulating within a lumen/balloon covering the transducer.

FIG. 21 shows cooling flow circulating between an inner and an outer balloon.

FIG. 22 is an ultrasound ablation element bounded by tandem occluding members.

FIG. 23 shows sector occlusion for targeted ablation and cooling.

FIG. 24 shows thermocouples incorporated into proximally slideable splines positioned over the outside of the balloon.

FIG. 25 shows thermocouples incorporated into splines fixed to the shaft but tethered to the distal end with an elastic member.

FIG. 26 shows thermocouples attached to the inside of the balloon, aligned with the ultrasound transducer.

FIG. 27 shows thermocouples positioned on the outside of the balloon, aligned with the ultrasound transducer, and routed across the wall and through the inside of the balloon.

FIGS. 28a-28c show the use of a slit in the elastic encapsulation of a thermocouple bonded to the outside of an elastic balloon that allows the thermocouple to become exposed during balloon inflation.

FIG. 29 shows thermocouples mounted on splines between two occluding balloons and aligned with the transducer

### DETAILED DESCRIPTION OF THE INVENTION

This Specification discloses various catheter-based systems and methods for treating the tissue containing nerve pathways in the outer vessel or extra-vascular tissue. The systems and methods are particularly well suited for treating renal vessels for control of hypertension. For this reason, the systems and methods will be described in this context.

Still, it should be appreciated that the disclosed systems and methods are applicable for use in treating other dysfunctions elsewhere in the body, which are not necessarily hypertension-related. For example, the various aspects of the invention have application in procedures where nerve modulation induces vessel dilation or constriction to aid ischemic stroke victims, or reduce the incidence of cerebral hemorrhage.

In general, this disclosure relates to the ability of the ultrasound to heat the tissue in order to cause it interrupt or remodulate nerve function.

For the purposes of interrupting or remodulating nerve function, it may be sufficient to deliver shock waves to the tissue such that the tissue matrix is mechanically disrupted (i.e, via cavitation), but not necessarily heated. This is another means by which ultrasound could be a more beneficial energy modality than others. The ultrasound could be delivered in high-energy MHz pulses or through lower kHz frequency levels.

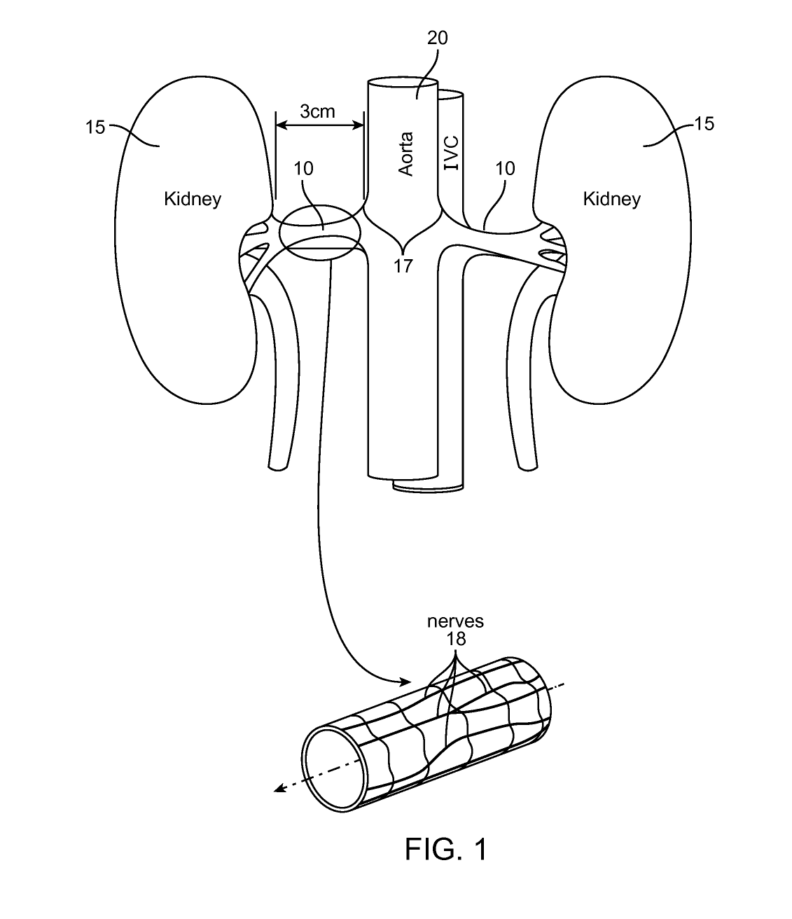

As FIG. 1 shows, the renal artery 10 is an approximately 3 cm long muscular tube that transports blood from the aorta 20 to the kidney 15.

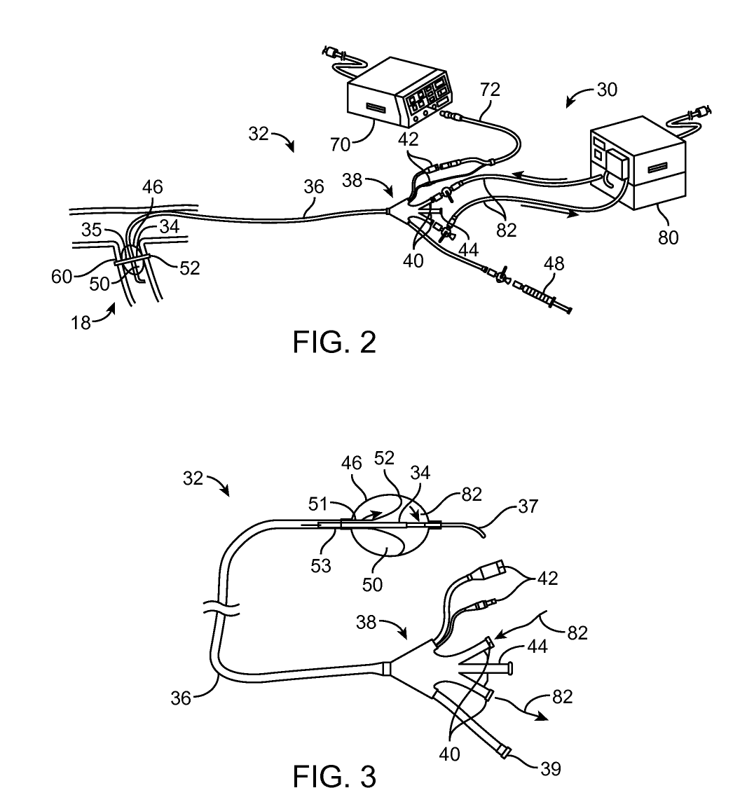

As shown in FIG. 2, the present invention relates to an ablation system 30 consisting of an ablation device 32 with an acoustic energy delivery element (ultrasound transducer) 34 mounted on the distal end of the catheter. The device is delivered intravascularly to the renal artery. The approach may be through the femoral artery as shown, or via a radial, carotid, or subclavian artery. Alternatively the approach could be via a femoral, jugular, or subclavian vein, when the device is to be positioned in a renal vein. The system 30 consists of the following key components:

1. A catheter shaft 36 with proximal hub 38 containing fluid ports 40, electrical connectors 42, and optional central guidewire lumen port 44.

2. An ultrasound transducer 34 that produces acoustic energy 35 at the distal end of the catheter shaft 36

3. An expandable balloon 46 operated with a syringe 48 used to create a fluid chamber 50 that couples the acoustic energy 35 to the tissue 60

4. Temperature sensor(s) 52 in the zone of energy delivery

5. An energy generator 70 and connector cable(s) 72 for driving the transducer and displaying temperature values

6. A fluid pump 80 delivering cooling fluid 82.

As shown in FIG. 3, the preferred embodiment of the ablation device consists of an ultrasound transducer 34 mounted within the balloon 46 near the distal end of an elongated catheter shaft 36. A proximal hub, or handle, 38 allows connections to the generator 70, fluid pump 80, and balloon inflation syringe 48. In other embodiments (not shown) the hub/handle 38 may provide a port for a guidewire and an actuator for deflection or spline deployment. The distal tip 39 is made of a soft, optionally preshaped, material such as low durometer silicone or urethane to prevent tissue trauma. The ultrasound transducer 34 is preferably made of a cylindrical ceramic PZT material, but could be made of other materials and geometric arrangements as are discussed in more detail below. Depending on performance needs, the balloon 46 may consist of a compliant material such as silicone or urethane, or a more non-compliant material such as nylon or PET, or any other material having a compliance range between the two. Temperature sensors 52 are aligned with the beam of acoustic energy 35 where it contacts the tissue. Various configurations of temperature monitoring are discussed in more detail below. The catheter is connected to an energy generator 70 that drives the transducer at a specified frequency. The optimal frequency is dependent on the transducer 34 used and is typically in the range of 7-10 MHz, but could be 1-40 MHz. The frequency may be manually entered by the user or automatically set by the generator 70 when the catheter is connected, based on detection algorithms in the generator. The front panel of the generator 70 displays power levels, delivery duration, and temperatures from the catheter. A means of detecting and displaying balloon inflation volume and/or pressure, and cooling flow rate/pressure may also be incorporated into the generator. Prior to ablation, the balloon 46 is inflated with a fluid such as saline or water, or an acoustic coupling gel, until it contacts the vessel over a length exceeding the transducer length. Cooling fluid 82 is used to minimize heat buildup in the transducer and keep the luminal surface temperatures in a safe range. In the preferred embodiment shown, cooling fluid 82 is circulated in through the balloon inflation lumen 51 and out through the central lumen 53 using a fluid pump 80. As described later, the circulation fluid may be routed through lumens different than the balloon lumen, requiring a separate balloon inflation port 39. Also, it may be advantageous to irrigate the outer proximal and/or distal end of the balloon for cooling. The path of this irrigating fluid could be from a lumen in the catheter and out through ports proximal and/or distal to the balloon, or from the inner lumen of a sheath placed over the outside of or alongside the catheter shaft.

In other embodiments (not shown) of the catheter, the central lumen 53 could allow passage of a guidewire (e.g., 0.035″) from a proximal port 44 out the distal tip 39 for atraumatic placement into the body. Alternatively, a monorail guidewire configuration could be used, where the catheter 30 rides on the wire just on the tip section 39 distal to the transducer 34. A central lumen with open tip configuration would also allow passage of an angioscope for visualization during the procedure. The catheter could also be fitted with a pull wire connected to a proximal handle to allow deflection to aid in placement in the renal vessel. This could also allow deflection of an angioscope in the central lumen. The balloon may also be designed with a textured surface (i.e., adhesive bulbs or ribs) to prevent movement in the inflated state. Finally, the catheter shaft or balloon or both could be fitted with electrodes that allow pacing and electrical signal recording within the vessel.

The above ablation device 32 is configured as an elongated catheter. A deflection mechanism and/or guidewire lumen may or may not be necessary. Of course, depending on the vessel being treated, the ablation device may be configured as a probe, or a surgically delivered instrument.

In use, the patient lies awake but sedated in a reclined or semi-reclined position. The physician inserts an introducer sheath through the skin and partially into the femoral artery. The introducer sheath may be sufficiently long to reach the renal vessel, or a subselective angiographic or guiding catheter may be used to cannulate the renal vessel. A guidewire may be placed into the renal vessel to aid in catheter advancement.

The physician preferably first conducts a diagnostic phase of the procedure, to image the vessel to be treated with contrast injected through the sheath angiographic catheter, or guiding catheter.

The physician then passes the ablation catheter through the introducer sheath or guiding catheter while visualizing using fluoroscopy.

The physician next begins the treatment phase of the procedure. The physician passes the catheter shaft 36 carrying the ultrasound transducer 34 through the introducer sheath or guiding catheter while visualizing using fluoroscopy. For the passage, the expandable balloon 46 is in its collapsed condition. The use of a pre-placed guidewire (0.014″-0.038″ diameter) is typically used for the whole device or at least a distal segment (˜5-30 cm) of the ablation device to track over into the vessel. Tracking over this guidewire may also be a soft tubular element which passes through the lumen and past the tip of catheter shaft 36. This tubular element may facilitate entry in to the renal artery by providing a smooth stiffness transition from the tip of the catheter to the guidewire. The tubular element and guidewire may be removed from the inside of catheter 36 to provide sufficient “runway” to position the transducer elements within the length of the renal artery. Use of a guidewire to track the ablation catheter may or may not be necessary. In some embodiments, deflection of the ablation device may be sufficient to steer the device into the renal vessel. Radiopaque markings on the catheter aid in device visualization in the vessel.

In FIG. 1, the targeted site is shown to be renal artery 10. The ostium 17 of the renal artery 10 with the aorta 20 may be targeted instead of, or in addition to, the main trunk of the renal artery.

Once located at the targeted site, the physician operates the syringe 48 to convey fluid or coupling gel into the expandable balloon 46. The balloon 46 expands to make intimate contact with the vessel surface, over a length longer than where the acoustic energy 35 impacts the tissue. The balloon is expanded to temporarily oppose the vessel wall, and to create a chamber 50 of fluid or gel through which the acoustic energy 35 couples to the tissue 60. The expanded balloon 46 also places the temperature sensors 52 in intimate contact with the vessel surface.

The physician commands the energy generator 70 to apply electrical energy to the ultrasound transducer 34. The function of the ultrasound transducer 34 is to then convert the electrical energy to acoustic energy 35.

The energy heats the tissue beyond the intima layer. The generator 70 displays temperatures sensed by the temperature sensors 80 to monitor the application of energy. The physician may choose to reduce the energy output of the generator 70 if the temperatures exceed predetermined thresholds. The generator 70 may also automatically shut off the power if temperature sensors 80 or other sensors in the catheter exceed safety limits.

Prior to energy delivery, it will most likely be necessary for the physician to make use of a fluid pump 80 to deliver cooling fluid 82 to keep the interior vessel temperature below a safe threshold. This is discussed in more detail later. The pump 80 may be integrated into the generator unit 70 or operated as a separate unit.

Preferably, for a region of the renal artery 10 or aorta 20, energy is applied to achieve tissue temperatures at the location of the nerves 18 in the range of 55° C. to 95° C. In this way, lesions can typically be created at depths ranging from one 1 mm beyond the intimal surface to as far as the extra-vascular structures 10. If applying energy from the vein, it may be desirable for the acoustic energy to heat one or both sides of the opposing renal artery tissue, with the intimal layers cooled by blood flow. This can require an acoustic penetration distance sufficient to heat at depths up to 15-20 mm. Typical acoustic energy densities range 10 to 100 W/cm2 as measured at the transducer surface. For focusing elements, the acoustic energy densities at the focal point are much higher.

It is desirable that the lesions possess sufficient volume to evoke tissue-healing processes accompanied by intervention of fibroblasts, myofibroblasts, macrophages, and other cells. The healing processes results in a contraction of tissue about the lesion, to further induce stretch related effects on the incident and surrounding nerves. Replacement of collagen by new collagen growth may also serve to remodel the vessel wall. Ultrasound energy typically penetrates deeper than is possibly by RF heating or thermal conduction alone.

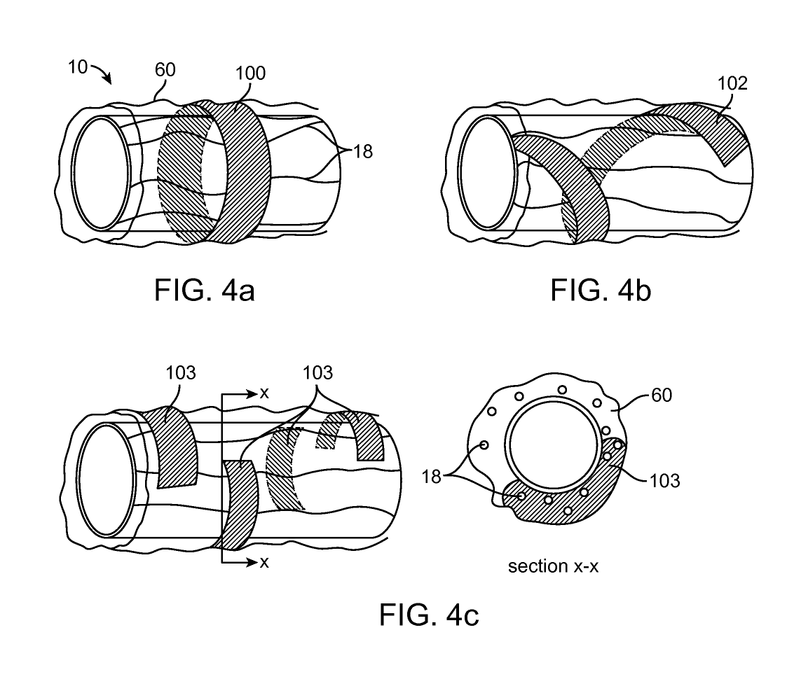

As shown in FIG. 4a, with a full circumferential output of acoustic energy 35 from ultrasound transducer 34, it is possible to create a completely circumferential lesion 100 in the tissue 60 of the renal vessel 18, at the ostium 17, or fully within the aorta 20. To create a more reliable result, it may be desirable to create a pattern of multiple circumferential lesions spaced axially along the length of the targeted treatment site in the renal artery 18, at the ostium 17, or fully within the aorta 20.

To limit the amount of tissue ablated, and still achieve the desired effect, it may be beneficial to spare and leave viable some circumferential sections of the vessel wall. This may help prevent severe stenosis in the vessel, maintain vessel elasticity, and/or blunt the remodulation effect. To this end, the ultrasound transducer 34 can be configured (embodiments of which are discussed in detail below) to emit ultrasound in discrete locations around the circumference and length of the vessel. Various lesion patterns such as 102 and 103 can be achieved. A preferred pattern (shown in FIG. 4c for the renal artery 10) comprises helically spaced pattern 103 of lesions about 5 mm apart, with the pattern 103 comprising preferably 4 (potential range 1-12) lesions. The width (measured along the length of the vessel) of each lesion could also fuse to achieve a continuous stepwise helical pattern mimicking that of 102 in FIG. 4b. Similarly, the longitudinal spacing of each lesion could be brought together to form a more closely fused fully circumferential lesion mimicking that of 100 in FIG. 4a. If only partial remodulation is desired, gaps around the circumference could be left to allow partial nerve conduction. The longitudinal length of the lesion pattern could range 2-40 mm, preferably 10-20 mm.

The physician can create a given ring pattern (either fully circumferential lesions or discrete lesions spaced around the circumference and/or vessel length) by expanding the balloon 46 with fluid or gel, pumping fluid 82 to cool the luminal tissue interface as necessary, and delivering electrical energy from the generator 70 to produce acoustic energy 35 to the tissue 60. The lesions in a given pattern can be formed simultaneously with the same application of energy, or one-by-one, or in a desired combination. Additional patterns of lesions can be created by advancing the ultrasound transducer 34 axially and/or rotationally, gauging the ring separation by the markings on the catheter shaft 36 and/or through fluoroscopic imaging of the catheter tip. In a given embodiment, the transducer may be moveable relative to the balloon, or in another embodiment, the entire balloon and transducer would be moved together to reposition. Other, more random or eccentric patterns of lesions can be formed to achieve the desired density of lesions within a given targeted site.

The catheter 32 can also be configured such that once the balloon 46 is expanded in place, the distal shaft 36 upon which the transducer 34 is mounted can be advanced axially within the balloon 46 that creates the fluid chamber 35, without changing the position of the balloon 46. Preferably, the temperature sensor(s) 52 move with the transducer 34 to maintain their position relative to the energy beam 35.

The distal catheter shaft 36 can also be configured with multiple ultrasound transducers 34 and temperature sensors 52 along the distal axis in the fluid chamber 35 to allow multiple lesions to be formed simultaneously or in any desired combination. They can also simply be formed one-by-one without having to adjust the axial position of the catheter 32.

To achieve certain heating effects, it may be necessary to utilize variations of the transducer, balloon, cooling system, and temperature monitoring. For instance, in order to prevent ablation of the interior surface of the vessel 10, it may be necessary to either (or both) focus the ultrasound under the surface, or sufficiently cool the surface during energy delivery. Temperature monitoring provides feedback as to the how well the tissue is being heated and cooled.

The following sections describe various embodiments of the ultrasound transducer 34 design, the mounting of the ultrasound transducer 34, cooling configurations, and means of temperature monitoring.

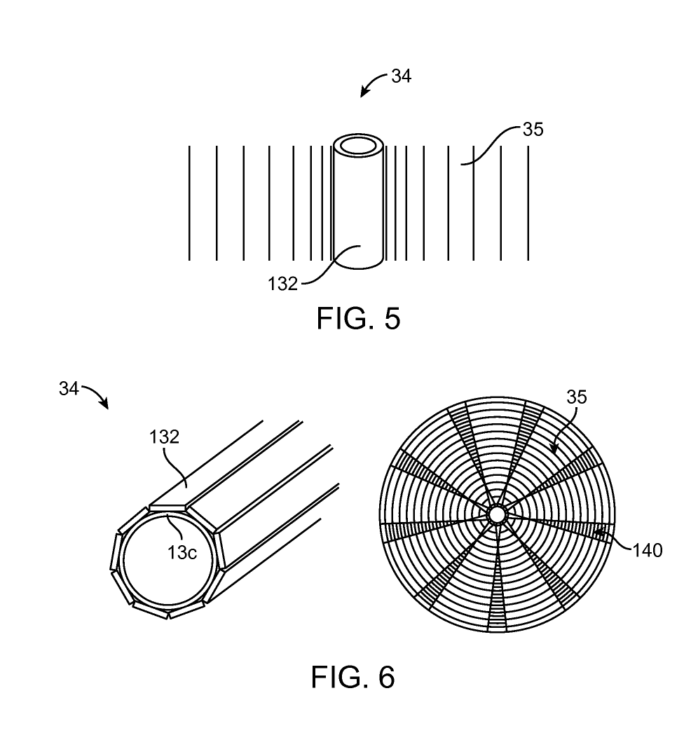

Ultrasound Transducer Design Configurations: In one preferred embodiment, shown in FIG. 5, the transducer 34 is a cylinder of PZT (e.g., PZT-4, PZT-8) material 130. The material is plated on the inside and outside with a conductive metal, and poled to “flip”, or align, the dipoles in the PZT material 130 in a radial direction. This plating 120 allows for even distribution of an applied potential across the dipoles. It may also be necessary to apply a “seed” layer (i.e., sputtered gold) to the PZT 130 prior to plating to improve plating adhesion. The dipoles (and therefore the wall of the material) stretch and contract as the applied voltage is alternated. At or near the resonant frequency, acoustic waves (energy) 35 emanate in the radial direction from the entire circumference of the transducer. The length of the transducer can be selected to ablate wide or narrow regions of tissue. The cylinder is 5 mm long in best mode, but could be 2-20 mm long. Inner diameter is a function of the shaft size on which the transducer is mounted, typically ranging from 1 to 4 mm. The wall thickness is a function of the desired frequency. An 8 MHz transducer would require about a 0.011″ thick wall.

In another embodiment of the transducer 34 design, illustrated in FIG. 6, multiple strips 132 of PZT 130 or MEMS (Micro Electro Mechanical Systems—Sensant, Inc., San Leandro, Calif.) material are positioned around the circumference of the shaft to allow the user to ablate selected sectors. The strips 132 generally have a rectangular cross section, but could have other shapes. Multiple rows of strips could also be spaced axially along the longitudinal axis of the device. By ablating specific regions, the user may avoid collateral damage in sensitive areas, or ensure that some spots of viable tissue remain around the circumference after energy delivery. The strips 132 may be all connected in parallel for simultaneous operation from one source, individually wired for independent operation, or a combination such that some strips are activated together from one wire connection, while the others are activated from another common connection. In the latter case, for example, where 8 strips are arranged around the circumference, every other strip (every 90°) could be activated at once, with the remaining strips (90° C. apart, but 45° C. from the previous strips) are activated at a different time. Another potential benefit of this multi-strip configuration is that simultaneous or phased operation of the strips 132 could allow for regions of constructive interference (focal regions 140) to enhance heating in certain regions around the circumference, deeper in the tissue. Phasing algorithms could be employed to enhance or “steer” the focal regions 140. Each strip 132 could also be formed as a curved x-section or be used in combination with a focusing lens to deliver multiple focal heating points 140 around the circumference.

The use of multiple strips 132 described above also allows the possibility to use the strips for imaging. The same strips could be used for imaging and ablation, or special strips mixed in with the ablation strips could be used for imaging. The special imaging strips may also be operated at a different frequency than the ablation strips. Since special imaging strips use lower power than ablation strips, they could be coated with special matching layers on the inside and outside as necessary, or be fitted with lensing material. The use of MEMs strips allows for designs where higher resolution “cells” on the strips could be made for more precise imaging. The MEMs design also allows for a mixture of ablation and imaging cells on one strip. Phasing algorithms could be employed to enhance the imaging.

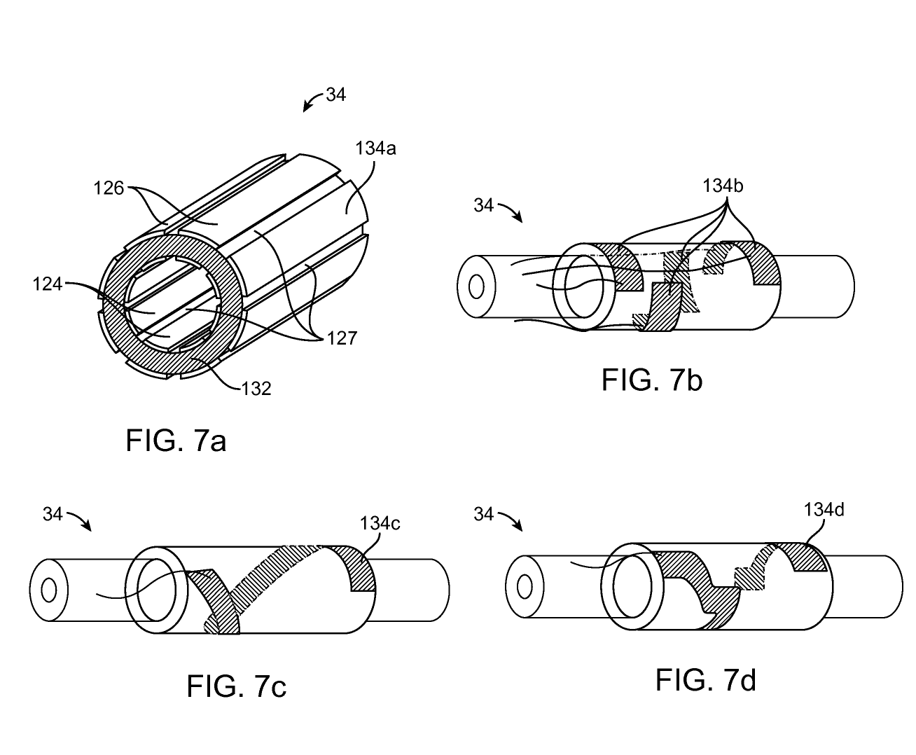

In another embodiment of the transducer 34 design, shown in FIG. 7a, a single cylindrical transducer 34 as previously described is subdivided into separate active longitudinal segments 134a arrayed around the circumference through the creation of discrete regions of inner plating 124 and outer plating 126. To accomplish this, longitudinal segments of the cylindrical PZT material 130 could be masked to isolate regions from one another during the plating process (and any seed treatment, as applicable). Masking may be accomplished by applying wax, or by pressing a plastic material against the PZT 130 surface to prevent plating adhesion. Alternatively, the entire inner and outer surface could be plated followed by selective removal of the plating (by machining, grinding, sanding, etc.). The result is similar to that shown in FIG. 10, with the primary difference being that the transducer is not composed of multiple strips of PZT 130, but of one continuous unit of PZT 130 that has different active zones electrically isolated from one another. Ablating through all at once may provide regions of constructive interference (focal regions 140) deeper in the tissue. Phasing algorithms could also be employed to enhance the focal regions 140. As shown in FIGS. 7b, 7c, and 7d, alternative active regions (134b, 134c, 134d, respectively) of the transducer can be constructed to allow energy delivery from discrete or continuous regions around both the circumference and length of the transducer structure (e.g., a continuous or step-wise helical pattern). Energy delivery in this pattern may allow complete interruption of nerve pathways around the vessel circumference while minimizing the risk of a focused stenosis in the vessel. Multiple continuous active regions oriented roughly parallel to one another could also be used to achieve other ablation patterns and/or modulation the heat generated during energy delivery.

As described above, this transducer 34 can also be wired and controlled such that the user can ablate specific sectors, or ablate through all simultaneously. Different wiring conventions may be employed. Individual “+” and “−” leads may be applied to each pair of inner 124 and outer 126 plated regions. Alternatively, a common “ground” may be made by either shorting together all the inner leads, or all the outer leads and then wiring the remaining plated regions individually.

Similarly, it may only be necessary to mask (or remove) the plating on either the inner 124 or the outer 126 layers. Continuous plating on the inner region 124, for example, with one lead extending from it, is essentially the same as shorting together the individual sectors. However, there may be subtle performance differences (either desirable or not) created when poling the device with one plating surface continuous and the other sectored.

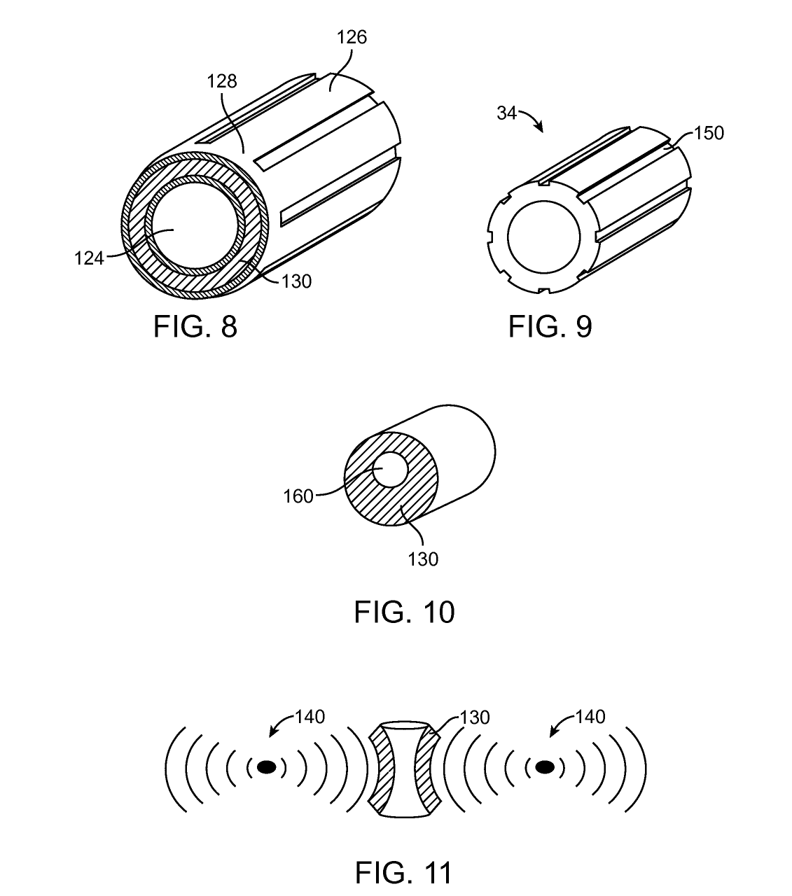

In addition to the concept illustrated in FIGS. 7a-d, it may be desirable to have a continuous plating ring 128 around either or both ends of the transducer 34, as shown in FIG. 8 (continuous plating shown on the proximal outer end only, with no discontinuities on the inner plating). This arrangement could be on either or both the inner and outer plating surface. This allows for one wire connection to drive the given transducer surface at once (the concept in FIGS. 7a-d would require multiple wire connections).

Another means to achieve discrete active sectors in a single cylinder of PZT is to increase or decrease the wall thickness (from the resonant wall thickness) to create non-resonant and therefore inactive sectors. The entire inner and outer surface can be then plated after machining. As illustrated in FIG. 9, channels 150 are machined into the transducer to reduce the wall thickness from the resonant value. As an example, if the desired resonant wall thickness is 0.0110″, the transducer can be machined into a cylinder with a 0.0080″ wall thickness and then have channels 150 machined to reduce the wall thickness to a non-resonant value (i.e., 0.0090″). Thus, when the transducer 34 is driven at the frequency that resonates the 0.0110″ wall, the 0.0090″ walls will be non-resonant. Or the transducer 34 can be machined into a cylinder with a 0.015″ wall thickness, for example, and then have selective regions machined to the desired resonant wall thickness of, say, 0.0110″. Some transducer PZT material is formed through an injection molding or extrusion process. The PZT could then be formed with the desired channels 150 without machining.

Another way to achieve the effect of a discrete zone of resonance is to machine the cylinder such that the central core 160 is eccentric, as shown in FIG. 10. Thus different regions will have different wall thicknesses and thus different resonant frequencies.

It may be desirable to simply run one of the variable wall thickness transducers illustrated above at a given resonant frequency and allow the non-resonant walls be non-active. However, this does not allow the user to vary which circumferential sector is active. As a result, it may be desirable to also mask/remove the plating in the configurations with variable wall thickness and wire the sectors individually.

In another method of use, the user may gain control over which circumferential sector is active by changing the resonant frequency. Thus the transducer 34 could be machined (or molded or extruded) to different wall thicknesses that resonate at different frequencies. Thus, even if the plating 122 is continuous on each inner 124 and outer 126 surface, the user can operate different sectors at different frequencies. This is also the case for the embodiment shown in FIG. 6 where the individual strips 132 could be manufactured into different resonant thicknesses. There may be additional advantages of ensuring different depths of heating of different sectors by operating at different frequencies. Frequency sweeping or phasing may also be desirable.

For the above transducer designs, longitudinal divisions are discussed. It is conceivable that transverse or helical divisions would also be desirable. Also, while the nature of the invention relates to a cylindrical transducer, the general concepts of creating discrete zones of resonance can also be applied to other shapes (planar, curved, spherical, conical, etc.). There can also be many different plating patterns or channel patterns that are conceivable to achieve a particular energy output pattern or to serve specific manufacturing needs.

Except where specifically mentioned, the above transducer embodiments have a relatively uniform energy concentration as the ultrasound propagates into the tissue. The following transducer designs relate to configurations that focus the energy at some depth. This is desirable to minimize the heating of the tissue at the inner vessel surface but create a lesion at some depth.

One means of focusing the energy is to apply a cover layer “lens” 170 (not shown) to the surface of the transducer in a geometry that causes focusing of the acoustic waves emanating from the surface of the transducer 34. The lens 170 is commonly formed out an acoustically transmissive epoxy that has a speed of sound different than the PZT material 130 and/or surrounding coupling medium. The lens 170 could be applied directly to the transducer, or positioned some distance away from it. Between the lens 170 and the transducer may be a coupling medium of water, gel, or similarly non-attenuating material. The lens could be suspended over (around) the transducer 34 within the balloon 46, or on the balloon itself.

In another embodiment, the cylindrical transducer 34 can be formed with a circular or parabolic cross section. As illustrated in FIG. 11, this design allows the beam to have focal regions 140 and cause higher energy intensities within the wall of the tissue.

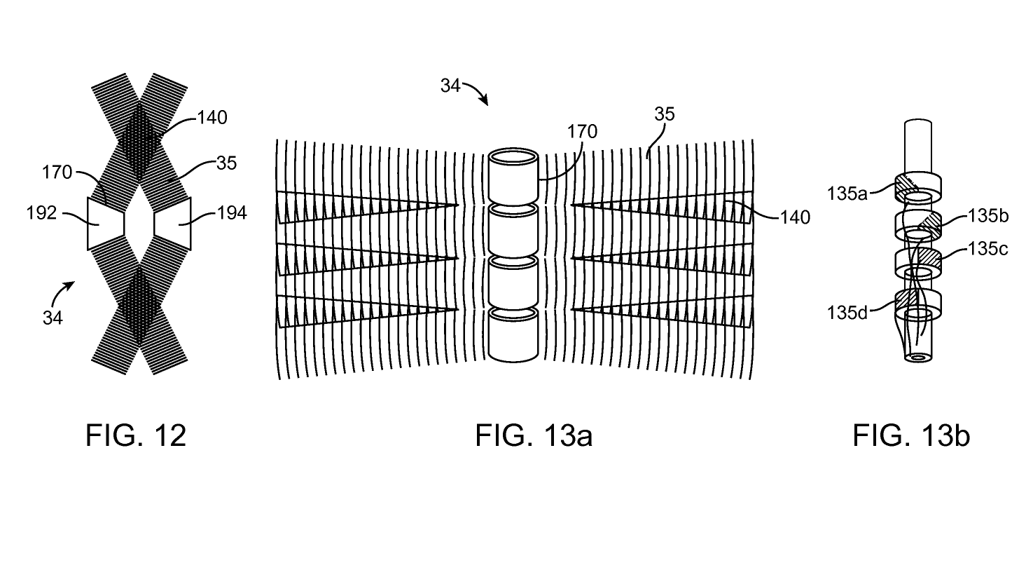

In another embodiment shown in FIG. 12, angled strips or angled rings (cones) allow forward and/or rear projection of ultrasound (acoustic energy 35). Rearward projection of ultrasound 35 may be particularly useful to heat the underside of the LES 18 or cardia 20 when the transducer element 34 is positioned distal to the LES 18. Each cone could also have a concave or convex shape, or be used with a lensing material 170 to alter the beam shape. In combination with opposing angled strips or cones (forward 192 and rearward 194) the configuration allows for focal zones of heating 140.

In another embodiment, shown in FIG. 13a, multiple rings (cylinders) of PZT transducers 34 would be useful to allow the user to change the ablation location without moving the catheter. Multiple rings may also allow more flex of the distal catheter tip, to enhance tracking into the vessel. Multiple rings also allows for regions of constructive/destructive interference (focal regions 140) when run simultaneously. Anytime multiple elements are used, the phase of the individual elements may be varied to “steer” the most intense region of the beam in different directions. Rings could also have a slight convex shape to enhance the spread and overlap zones, or a concave shape to focus the beam from each ring. Pairs of opposing cones or angled strips (described above) could also be employed. Each ring could also be used in combination with a lensing material 170 to achieve the same goals. As shown in FIG. 13b, each ring could also have only partial sectors 135a-d active (via selective plating, or thickness variation controlling the resonant frequency), such that different quadrants can be activated along the total length of the rings.

Transducer Mounting: One particular challenge in designing transducers that deliver significant power (approximately 10 acoustic watts per cm² at the transducer surface, or greater) is preventing the degradation of adhesives and other heat/vibration sensitive materials in proximity to the transducer. If degradation occurs, materials under or over the transducer can delaminate and cause voids that negatively affect the acoustic coupling and impedance of the transducer. In cases where air backing of the transducer is used, material degradation can lead to fluid infiltration into the air space that will compromise transducer performance. Some methods of preventing degradation are described below.

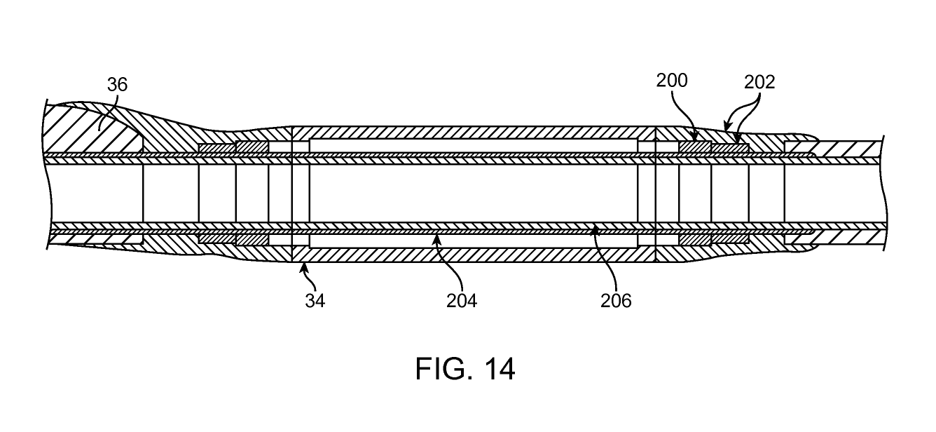

In FIG. 14, a preferred means of mounting the transducer 34 is to securely bond and seal (by welding or soldering) the transducer to a metal mounting member 200 that extends beyond the transducer edges. Adhesive attachments 202 can then be made between the mounting member 200 extensions remote to the transducer 34 itself. The mounting member(s) can provide the offsets from the underlying mounting structure 206 necessary to ensure air backing between the transducer 34 and the underlying mounting structure 206. One example of this is shown in FIG. 14 where metal rings 200 are mounted under the ends of the transducer 34. The metal rings 200 could also be attached to the top edges of the transducer 34, or to a plated end of the transducer. It may also be possible to mechanically compress the metal rings against the transducer edges. This could be accomplished through a swaging process or through the use of a shape-memory material such as nitinol. It may also be possible to use a single metal material under the transducer as the mounting member 200 that has depressions (i.e. grooves, holes, etc.) in the region under the transducer to ensure air backing. A porous metal or polymer could also be placed under the transducer 34 (with the option of being in contact with the transducer) to provide air backing.

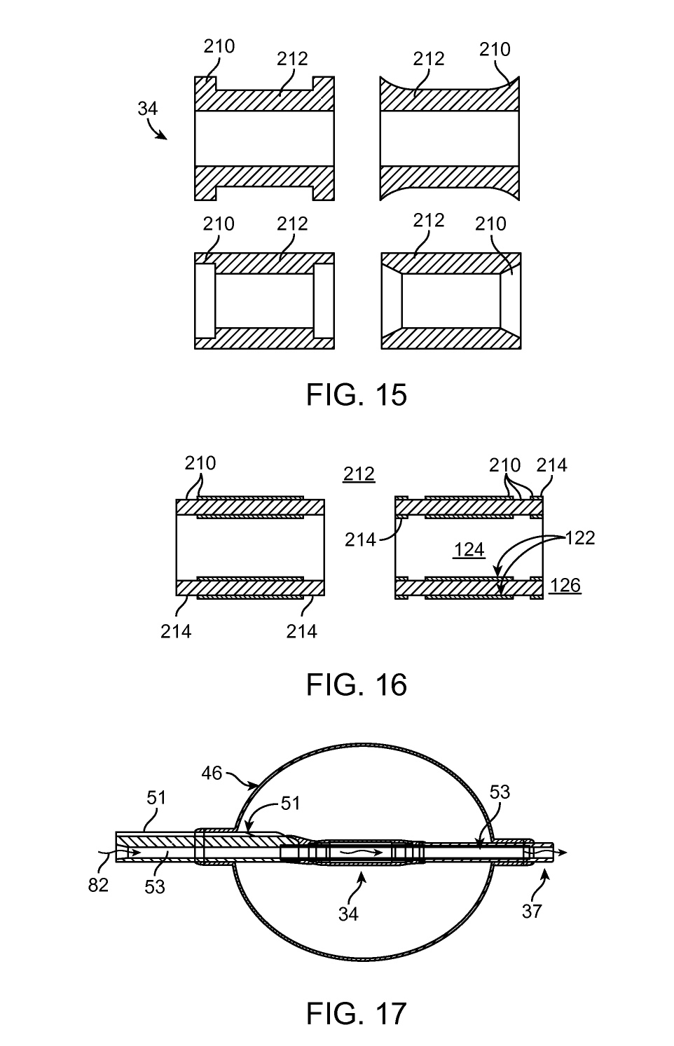

In FIG. 15, another means of mounting the transducer 34 is to form the transducer 34 such that non-resonating portions 210 of the transducer 34 extend away from the central resonant section 212. The benefit is that the non-resonant regions 210 are integral with the resonant regions 212, but will not significantly heat or vibrate such that they can be safely attached to the underlying mounting structure 206 with adhesives 202. This could be accomplished by machining a transducer 34 such that the ends of the transducer are thicker (or thinner) than the center, as shown in FIG. 15.

As shown in FIG. 16, another option is to only plate the regions of the transducer 34 where output is desired, or interrupt the plating 122 such that there is no electrical conduction to the mounted ends 214 (conductor wires connected only to the inner plated regions).

The embodiments described in FIGS. 14-16 can also be combined as necessary to optimize the mounting integrity and transducer performance.

Cooling Design Configurations: Cooling flow may be necessary to 1) Prevent the transducer temperature from rising to levels that may impair performance, and 2) Prevent the inner vessel layer(s) (e.g., intima and/or media) from heating to the point of irreversible damage. The temperature at the inner vessel layer(s) should be maintained between 5° C. and 50° C., preferably 20° C.-40° C. during acoustic energy delivery. The following embodiments describe the various means to meet these requirements.

FIG. 17 shows cooling fluid 82 being passed through a central lumen 53 and out the distal tip 37 to prevent heat buildup in the transducer 34. The central column of fluid 82 serves as a heat sink for the transducer 34.

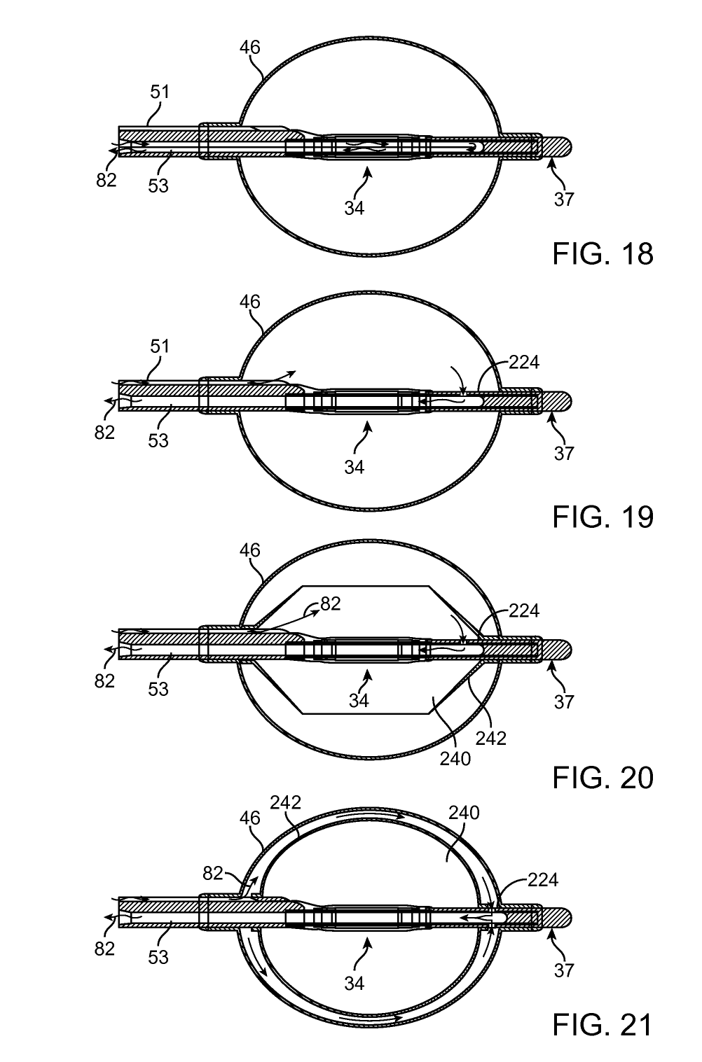

FIG. 18 is similar to FIG. 17 except that the fluid 82 is recirculated within the central lumen 53 (actually a composition of two or more lumens), and not allowed to pass out the distal tip 37.

FIG. 19 (also shown a part of the preferred embodiment of FIG. 2) shows the fluid circulation path involving the balloon itself. The fluid enters through the balloon inflation lumen 51 and exits through one or more ports 224 in the central lumen 53, and then passes proximally out the central lumen 53. The advantage of this embodiment is that the balloon 46 itself is kept cool, and draws heat away from the inner layer(s) of the vessel. Pressure of the recirculating fluid 82 would have to be controlled within a tolerable range to keep the balloon 46 inflated the desired amount. Conceivably, the central lumen 53 could be the balloon inflation lumen, with the flow reversed with respect to that shown in FIG. 19. Similarly, the flow path does not necessarily require the exit of fluid in the central lumen 53 pass under the transducer 34—fluid 82 could return through a separate lumen located proximal to the transducer.

In another embodiment (not shown), the balloon could be made from a porous material that allowed the cooling fluid to exit directly through the wall of the balloon. Examples of materials used for the porous balloon include open cell foam, ePTFE, porous urethane or silicone, or a polymeric balloon with laser-drilled holes.

FIG. 20 shows the encapsulation of the transducer 34 within another lumen 240. This lumen 240 is optionally expandable, formed from a compliant or non-compliant balloon material 242 inside the outer balloon 46 (the lumen for inflating the outer balloon 46 is not shown). This allows a substantial volume of fluid to be recirculated within the lumen 240 without affecting the inflation pressure/shape of the outer balloon 46 in contact with the luminal surface. Allowing a substantial inflation of this lumen decreases the heat capacity of the fluid in the balloon in contact with the luminal surface and thus allows for more efficient cooling of the inner vessel layer(s). Fluid 82 could also be allowed to exit the distal tip. It can also be imagined that a focusing lens material 170 previously described could be placed on the inner or outer layer of the lumen material 242 surrounding the transducer 34.

As is shown in FIG. 21, there can be an outer balloon 46 that allows circulation over the top of the inner balloon 242 to ensure rapid cooling at the interface. To ensure flow between the balloons, the inner balloon 242 can be inflated to a diameter less than the outer balloon 46. Flow 82 may be returned proximally or allowed to exit the distal tip. Another version of this embodiment could make use of raised standoffs 250 (not shown) either on the inside of the outer balloon 46 or the outside of the inner balloon 242, or both. The standoffs 250 could be raised bumps or splines. The standoffs 250 could be formed in the balloon material itself, from adhesive, or material placed between the balloons (i.e., plastic or metal mandrels). The standoffs 250 could be arranged longitudinally or circumferentially, or both. While not shown in a figure, it can be imagined that the outer balloon 46 shown in FIG. 21 may only need to encompass one side (i.e., the proximal end) of the inner balloon, allowing sufficient surface area for heat convection away from the primary (inner) balloon 242 that in this case may be in contact with the tissue.

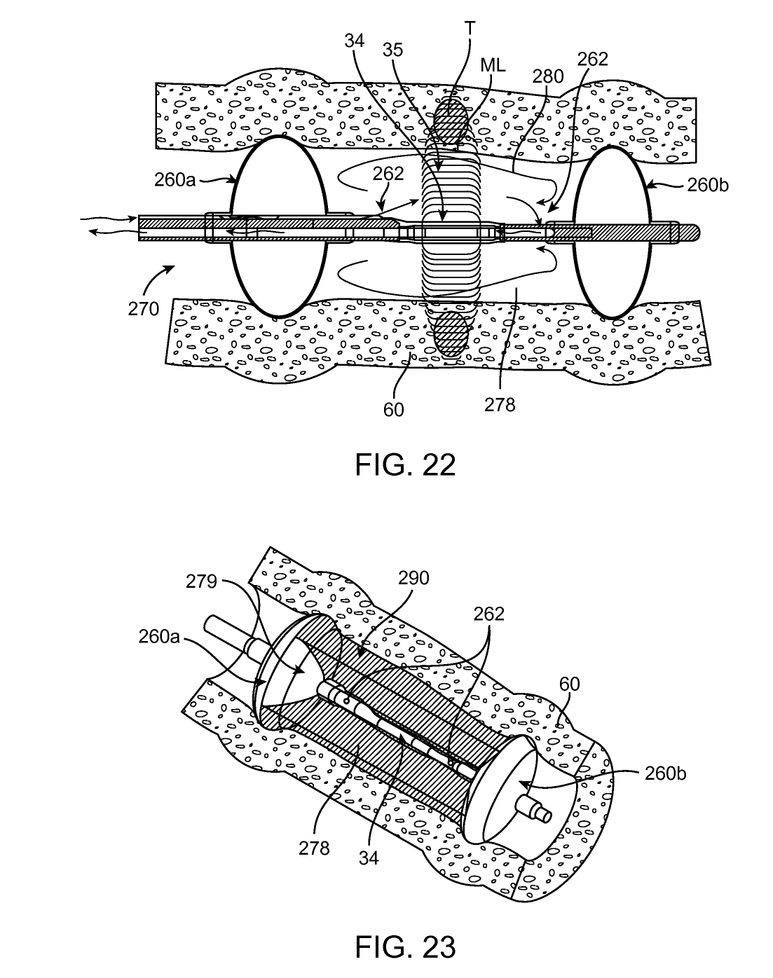

In another embodiment, illustrated in FIG. 22, occluding members 260 are positioned proximal (260a) and distal (260b) to the transducer element for occluding the vessel lumen 270. The occluding members 260 may also serve to dilate the vessel to a desired level. The occluding members 260 are capable of being expanded from a collapsed position (during catheter delivery) for occlusion. Each occluding member 260 is preferably an inflatable balloon, but could also be a self-expanding disk or foam material, or a wire cage covered in a polymer, or combination thereof. To deploy and withdraw a non-inflatable occluding member, either a self-expanding material could be expanded and compressed when deployed out and back in a sheath, or the occluding member could be housed within a braided or other cage-like material that could be alternatively cinched down or released using a pull mechanism tethered to the proximal end of the catheter 30. It may also be desirable for the occluding members 260 to have a “textured” surface to prevent slippage of the device. For example, adhesive spots could be applied to the outer surface of the balloon, or the self-expanding foam could be fashioned with outer ribs.

With the occluding members 260 expanded against the inner lumen, the chamber 278 formed between the balloons is then filled with a fluid or gel 280 that allows the acoustic energy 35 to couple to the tissue 60. To prevent heat damage to the inner layer(s) of the tissue lumen 270, the fluid/gel 280 may be chilled and/or recirculated. Thus with cooling, the lesion formed within the tissue 60 is confined inside the tissue wall and not formed at the inner surface. This cooling/coupling fluid 280 may be routed into and out of the space between the occluding members with single entry and exit port, or with a plurality of ports. The ports can be configured (in number, size, and orientation) such that optimal or selective cooling of the inner vessel layer(s) is achieved. Note also that cooling/coupling fluid 280 routed over and/or under the transducer 34 helps keep the transducer cool and help prevent degradation in performance.

The transducer element(s) 34 may be any of those previously described. Output may be completely circumferential or applied at select regions around the circumference. It is also conceivable that other energy sources would work as well, including RF, microwave, laser, and cryogenic sources.

In the case where only certain sectors of tissue around the circumference are treated, it may be desirable to utilize another embodiment, shown in FIG. 23, of the above embodiment shown in FIG. 22. In addition to occluding the proximal and distal ends, such a design would use a material 290 to occlude regions of the chamber 278 formed between the distal and proximal occluding members 260. This would, in effect, create separate chambers 279 around the circumference between the distal and proximal occluding members 260, and allow for more controlled or greater degrees of cooling where energy is applied. The material occluding the chamber could be a compliant foam material or an inflatable balloon material attached to the balloon and shaft. The transducer would be designed to be active only where the chamber is not occluded.

Temperature Monitoring: The temperature at the interface between the tissue and the balloon may be monitored using thermocouples, thermistors, or optical temperature probes. Although any one of these could be used, for the illustration of various configurations below, only thermocouples will be discussed. The following concepts could be employed to measure temperature.

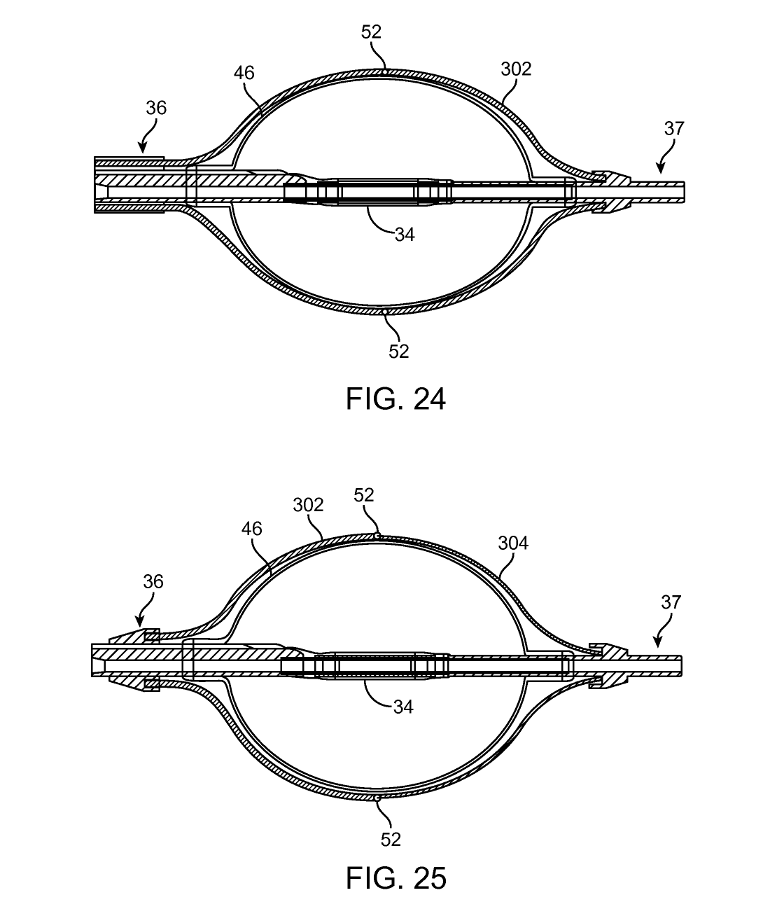

In one embodiment shown in FIG. 24, one or more splines 302, supporting one or more temperature sensors 52 per spline, run longitudinally over the outside of the balloon 46. On each spline 302 are routed one or more thermocouple conductors (actually a pair of wires) 306. The temperature sensor 52 is formed at the electrical junction formed between each wire pair in the conductor 306. The thermocouple conductor wires 306 could be bonded straight along the spline 302, or they could be wound or braided around the spline 302, or they could be routed through a central lumen in the spline 302.

At least one thermocouple sensor 52 aligned with the center of the ultrasound beam 35 is desired, but a linear array of thermocouple sensors 52 could also be formed to be sure at least one sensor 52 in the array is measuring the hottest temperature. Software in the generator 70 may be used to calculate and display the hottest and/or coldest temperature in the array. The thermocouple sensor 52 could be inside or flush with the spline 302; however, having the sensor formed in a bulb or prong on the tissue-side of the spline 302 is preferred to ensure it is indented into the tissue. It is also conceivable that a thermocouple placed on a slideable needle could be used to penetrate the tissue and measure the subintimal temperature.

Each spline 302 is preferably formed from a rigid material for adequate tensile strength, with the sensors 52 attached to it. Each individual spline 302 may also be formed from a braid of wires or fibers, or a braid of the thermocouple conductor wires 306 themselves. The splines 302 preferably have a rectangular cross section, but could also be round or oval in cross section. To facilitate deployment and alignment, the splines 302 may be made out a pre-shaped stainless steel or nitinol metal. One end of the spline 302 would be fixed to the catheter tip 37, while the proximal section would be slideable inside or alongside the catheter shaft 36 to allow it to move with the balloon 46 as the balloon inflates. The user may or may not be required to push the splines 302 (connected to a proximal actuator, not shown) forward to help them expand with the balloon 46.

The number of longitudinal splines could be anywhere from one to eight. If the transducer 34 output is sectored, the splines 302 ideally align with the active transducer elements.

In a related embodiment, a braided cage (not shown) could be substituted for the splines 302. The braided cage would be expandable in a manner similar to the splines 302. The braided cage could consist of any or a combination of the following: metal elements for structural integrity (i.e., stainless steel, nitinol), fibers (i.e., Dacron, Kevlar), and thermocouple conductor wires 306. The thermocouple sensors 52 could be bonded to or held within the braid. For integrity of the braid, it may be desirable for the thermocouple conductors 306 to continue distal to the thermocouple junction (sensor) 52. The number structural elements in the braid may be 4 to 24.

In another embodiment shown in FIG. 25, a design similar to the embodiment above is used, except the distal end of the spline 302 is connected to a compliant band 304 that stretches over the distal end of the balloon as the balloon inflates. The band 304 may be formed out of a low durometer material such as silicone, urethane, and the like. It may also be formed from a wound metal spring. The spline 302 proximal to the balloon may then be fixed within the catheter shaft 36. Of course the arrangement could be reversed with the spline 302 attached to the distal end of the balloon 46, and the compliant band 304 connected to the proximal shaft 36.

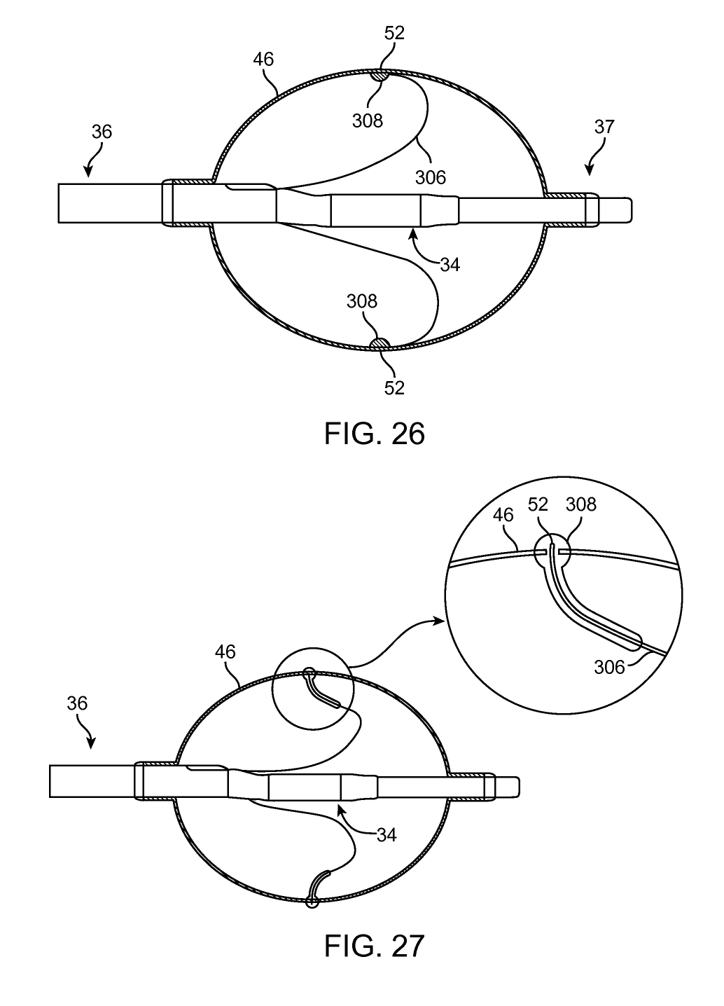

In another embodiment shown in FIG. 26, the sensors 52 are bonded with adhesive 308 to the inside of the balloon (in the path of the ultrasound beam 35). The adhesive 308 used is ideally a compliant material such as silicone or urethane if used with a compliant balloon. It may also be a cyanoacrylate, epoxy, or UV cured adhesive. The end of the conductor wire 306 at the location of the sensor 52 is preferably shaped into a ring or barb or the like to prevent the sensor from pulling out of the adhesive. Multiple sensors 52 may be arranged both circumferentially and longitudinally on the balloon 46 in the region of the ultrasound beam 35. Thermocouple conductor wires 306 would have sufficient slack inside the balloon 46 to expand as the balloon inflates.

In another embodiment (not shown), the thermocouple conductor wires are routed longitudinally through the middle of the balloon wall inside preformed channels.

In another embodiment shown in FIG. 27, the thermocouple sensors 52 are bonded to the outside of the balloon 46, with the conductor wires 306 routed through the wall of the balloon 46, in the radial direction, to the inside of the balloon 46 and lumens in the catheter shaft 36. The conductor wires 306 would have sufficient slack inside the balloon to expand as the balloon inflates. To achieve the wire routing, a small hole is punched in the balloon material, the conductor wire routed through, and the hole sealed with adhesive. The conductor wire could be coated in a material that is bondable with the balloon (i.e., the balloon material itself, or a compatible adhesive 308 as described for FIG. 26) prior to adhesive bonding to help ensure a reliable seal.

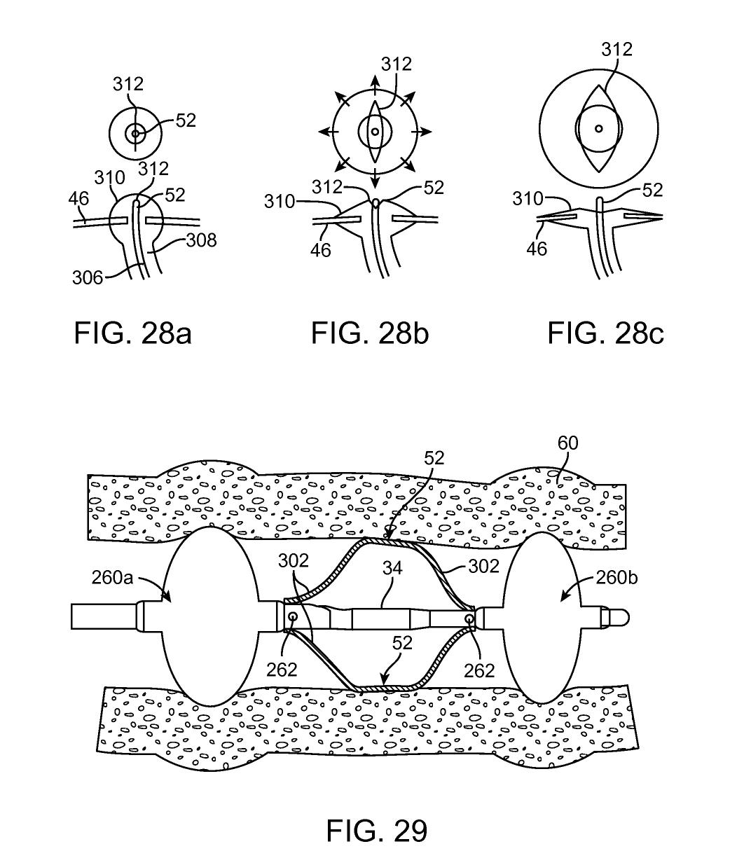

In another embodiment shown in FIGS. 28a-c, the thermocouple sensors 52 mounted on the outer surface of the balloon (regardless of how the wires 306 are routed) are housed in raised bulbs 310 of adhesive 308 (or a molded section of the balloon material itself) that help ensure they are pushed into the tissue, allowing more accurate tissue temperature measurement that is less susceptible to the temperature gradient created by the fluid in the balloon. For compliant balloons, a stiff exposed sensor 52 could be housed in a bulb of compliant material with a split 312. As the balloon 46 inflates, the split 312 in the bulb 210 opens and exposes the sensor 52 to the tissue. As the balloon 46 deflates, the bulb 310 closes back over the sensor 52 and protects it during catheter manipulation in the body.

In another embodiment (not shown), an infrared sensor pointed toward the heat zone at the balloon-tissue interface could be configured inside the balloon to record temperatures in a non-contact means.

For the embodiments described in either FIG. 22 or FIG. 24 above, it may also be desirable to monitor the temperature of the tissue during energy delivery.

This would be best accomplished through the use of thermocouples aligned with the ultrasound beam emanating from the transducer. Each thermocouple would monitor the temperature of the luminal surface to ensure that the appropriate amount of power is being delivered. Power can be decreased manually or though a feedback control mechanism to prevent heat damage to the inner vessel layer(s), or the power can be increased to a predetermined safe inner surface temperature rise to ensure adequate power is being delivered to the outer vessel layer and extra-vascular structures.

As shown in FIG. 29, the thermocouple sensors 52 could be mounted on splines 302 similar in design, construction, and operation to those described previously. In this configuration, the splines 302 are expanded against the tissue without the use of an interior balloon. They are deployed before, during, or after the occlusion members 260 are expanded. The braided cage configuration described above may also be used.

In another embodiment (not shown), the splines 302 or braided cage containing the thermocouple sensors 52 could span over the top of either or both expandable occlusive members 260. If the occlusive members 260 are balloons, the balloons act to expand the cage outward and against the tissue. If the occlusive members 206 are made from a self-expanding foam or disk material, the cage can be used to contain the occlusive material 206 during advancement of the catheter by holding the individual components of the cage down against the shaft under tension. Once positioned at the site of interest, the cage can be manually expanded to allow the occlusive members 260 to self-expand.

What is claimed is:

1. A method for remodeling outer vascular and/or extra-vascular tissue containing nerve conduction pathways, said method comprising:
providing a catheter having a proximal end, a distal end, a cylindrical transducer near the distal end, and a balloon surrounding the transducer,
positioning the catheter to locate the balloon at a target site in a blood vessel of a patient;
inflating the balloon with an acoustically transmissive medium, wherein the balloon is engaged against a vessel wall;
cooling the vessel wall where the balloon is engaged; and
energizing the transducer to transmit acoustic energy through the acoustically transmissive fluid to vascular nerves under conditions selected to induce nerve remodeling in at least a portion of the tissue circumferentially surrounding the balloon in the blood vessel.

2. A method as in claim 1, wherein the acoustic energy is produced under conditions which at least shrink the tissue.

3. A method as in claim 1, wherein the acoustic energy is produced under conditions which at least induce collagen formation in the tissue.

4. A method as in claim 1, wherein the acoustic energy is produced under conditions which at least cause cavitation in the tissue.

5. A method as in claim 1, wherein the acoustic energy is produced under conditions which at least interrupt nerve pathways in the tissue.

6. A method as in claim 1, wherein the acoustic energy is produced under conditions which at least modify nerve pathways in the tissue.

7. A method as in claim 1, wherein the transducer is energized to produce acoustic energy in the range from 10 W/cm² to 100 W/cm².

8. A method as in claim 1, wherein the transducer is energized at a duty cycle from 10% to 100%.

9. A method as in claim 1, wherein the transducer is energized under conditions which heat the nerves to a temperature in the range from 55° C. to 95° C.

10. A method as in claim 1, further comprising cooling the blood vessel intima surface while tissue beneath the surface is heated.

11. A method as in claim 1, wherein positioning the transducer comprises introducing a catheter which carries the transducer into the vessel.

12. A method as in claim 1, further comprising moving the transducer relative to the balloon(s) in order to focus or scan the acoustic energy axially on the blood vessel.

13. A method as in claim 1, wherein the acoustically transmissive medium is cooled to cool the blood vessel intima surface.

14. A method as in claim 1, wherein the acoustically transmissive medium is circulated in and out of the balloon to cool the blood vessel intima surface.

15. A method as in claim 1, further comprising monitoring temperature at the blood vessel intima surface.

16. A method as in claim 1, wherein the temperature at in the blood vessel intima is kept below 50° C. during acoustic energy delivery.

17. A method as in claim 1, further comprising monitoring temperature below the blood vessel intima surface.

18. A method as in claim 1, wherein energizing comprises focusing the acoustic energy beneath the blood vessel intima surface.

19. A method as in claim 18, wherein the transducer comprised a phased array.

20. A method as in claim 19, wherein the phased array is selectively energized to focus the acoustic energy at one or more desired locations in the tissue surrounding the vessel.

21. A method as in claim 1, wherein the vessel is a renal vessel and the patient suffers from hypertension.

22. A method as in claim 21, wherein the acoustic energy remodels the nerves surrounding the renal artery.

23. A method as in claim 1, wherein the vessel is a vessel of the neck or head, and the patient suffers from a stroke.

24. A method as in claim 23, where the vessel is a carotid artery.

25. Apparatus for remodeling the outer vascular and/or extra-vascular tissue containing nerve conduction pathways, said apparatus comprising:
a catheter adapted to be intravascularly introduced into a blood vessel;
an inflatable balloon disposed near a distal end of the catheter; and
means for inflating the balloon with an acoustically transmissive medium;
a means to cool the luminal surface of the blood vessel
a cylindrical transducer on the catheter inside the balloon, wherein said transducer has a length, an outer surface, and an inner surface wherein the transducer can be energized to deliver acoustic energy to remodel the outer vascular and/or extra-vascular tissue containing nerve conduction pathways when said balloon is inflated within the blood vessel.

26. Apparatus as in claim 25, wherein the transducer is positioned coaxially with the balloon.

27. Apparatus as in claim 25, further comprising means for cooling the acoustically transmissive medium.

28. Apparatus as in claim 25, further comprising means for circulating the acoustically transmissive medium in an out of the balloon to cool the vessel surface.

29. Apparatus as in claim 25, further comprising means for measuring temperature at or beneath the luminal wall.

30. Apparatus as in claim 25, further comprising means to axially translate the transducer relative to the catheter.

31. Apparatus as in claim 25, wherein the transducer comprises a phased array.
